(19) United States
(12) Patent Application Publication
(10) Pub. No.: US 2011/0243869 A1
(43) Pub. Date: Oct. 6, 2011

(54) WAX-FREE COSMETIC COMPOSITIONS CONTAINING A LATEX AND A FILLER

(75) Inventors: Dhaneswar Brijlall, Mercerville, NJ (US); Lilavati Patel, Edison, NJ (US); Bruno Bavouzet, Gentilly (FR)

(73) Assignee: L'OREAL, Paris (FR)

(21) Appl. No.: 13/076,939

(22) Filed: Mar. 31, 2011

Related U.S. Application Data
(60) Provisional application No. 61/319,731, filed on Mar. 31, 2010.

(51) Int. Cl. A61K 8/72; A61Q 1/00

(52) U.S. Cl. 424/70.6

(57) ABSTRACT

Disclosed are cosmetic compositions containing from about 10% to about 60% of a latex, and from about 10% to about 60% of a filler; wherein the ratio of latex to filler is from about 3.5:1 about 1:1.5, and the composition is free of wax. Also disclosed are methods for making the cosmetic compositions, wherein the entire process is carried out a temperature of about 30° C. or less. Further disclosed are methods of making up or enhancing the appearance of a keratinous substance comprising applying the cosmetic composition to the keratinous substance.

### CROSS-REFERENCE TO RELATED APPLICATIONS

This application claims the benefit of the filing date of U.S. Provisional Patent Application No. 61/319,731, filed Mar. 31, 2010, the disclosure of which is hereby incorporated herein by reference.

### BACKGROUND OF THE INVENTION

The present invention generally relates to novel wax-free cosmetic compositions including mascara, and a method of use, including a method of making-up eyes. More particularly, the present invention relates to wax-free mascara compositions having excellent volume, non-flaking, and tubing properties, which can not be achieved by conventional mascaras containing waxes.

Mascara compositions commonly contain waxes. Waxes are not only expensive, they are also not readily compatible with other ingredients. Moreover, waxes must be melted and do not readily disperse in the other ingredients of cosmetic compositions. The melting and dispersion of waxes require specialized equipment, additional manufacturing steps, and a large amount of energy. For all of these reasons, waxes add significantly to the cost of production of conventional mascara.

Therefore, it is desirable to provide a cosmetic composition that has excellent long wear, non-flaking, volume, and tubing properties without the need to use waxes.

### SUMMARY OF THE INVENTION

A first aspect of the present invention is directed to a wax-free cosmetic composition, including mascara, containing about 10% to about 60% of a latex and about 10% to about 60% of a filler, wherein the ratio of latex to filler is from about 3.5:1 about 1:1.5, and the composition is free of wax, wherein the percentages are by weight based on the total weight of the cosmetic composition.

A second aspect of the present invention is directed to a method of preparing a wax-free cosmetic composition, including mascara, by mixing together about 10% to about 60% of a latex and about 10% to about 60% of a filler, wherein the ratio of latex to filler is from about 3.5:1 about 1:1.5, and the composition is free of wax, wherein the percentages are by weight based on the total weight of the cosmetic composition.

A third aspect of the present invention is directed to a method of making up or enhancing the appearance of a keratinous substance by applying to the keratinous tissue a wax-free cosmetic composition, including mascara, containing about 10% to about 60% of a latex and about 10% to about 60% of a filler, wherein the ratio of latex to filler is from about 3.5:1 about 1:1.5, and the composition is free of wax, wherein the percentages are by weight based on the total weight of the cosmetic composition.

It has been surprisingly found that a composition according to an embodiment of the present invention has excellent long wear, non-flaking, volume, and tubing properties without the need for wax.

### DETAILED DESCRIPTION

Other than in the operating examples, or where otherwise indicated, all numbers expressing quantities of ingredients and/or reaction conditions are to be understood as being modified in all instances by the term “about,” meaning within 10% to 15% of the indicated number.

“Keratinous tissue”, as used herein, includes but is not limited to skin, hair, and nails.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C. “Non-volatile”, as used herein, means having a flash point of greater than about 100° C.

As used herein, the expressions “at least one”, “a”, and “an” mean one or more and thus include individual components as well as mixtures/combinations.

“Long wear” compositions as used herein, refer to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Flaking,” as used herein, refers to the flexibility and substantivity of the cosmetic composition. Large amounts of flaking indicate that the composition is inflexible and/or does not adhere well to the keratinous tissue. Thus, a composition with good non-flaking properties is one that exhibits little or no flaking. Flaking may be evaluated by any method known in the art for evaluating such properties. For example, flaking may be evaluated by a test involving the application of a composition to human eyelashes or hair followed by brushing the eyelashes or hair after a period of drying. Any flakes produced may be observed and the degree of flaking evaluated. Further, these characteristics may be evaluated with respect to other compositions, such as commercially available compositions.

“Volume,” as used herein, refers to the apparent build up of the composition on a keratinous tissue. In the example of a mascara, as volume increases each eyelash is defined and stands out to the eye, creating the desirable impression of longer and fuller lashes. A composition that produces greatly increased thickness is considered to have good volume properties. Volume may be evaluated by any method known in the art for evaluating such properties. For example, volume may be evaluated by a test involving first measuring the thickness of human eyelashes or hair. The composition is then applied to the eyelashes or hair. After a period of drying, the thickness of the eyelashes or hair is once again measured. The difference in volume from before and after treatment is then determined. Further, these characteristics may be evaluated with respect to other compositions, such as commercially available compositions.

“Tubing,” as used herein, refers to the propensity of the composition to roll up and off of the keratinous tissue when being removed. A composition that quickly and easily rolls up and off of the keratinous tissue when removal is desired is considered to have good tubing properties. Tubing may be evaluated by any method known in the art for evaluating such properties. For example, tubing may be evaluated by a test involving the application of a composition to human eyelashes or hair. After a period of drying eyelashes or hair are run under hot water. The lashes or hair are then laid down on paper and cardboard placed on top. The lashes or hair are then dragged out from below the cardboard and the degree of removal (tubing) is evaluated. Further, these characteristics may be evaluated with respect to other compositions, such as commercially available compositions.

### Latex

According to the present invention, compositions comprising a latex are provided. The latexes useful in the present invention are particles of film-forming polymer in an aqueous dispersion. Representative examples of suitable latexes include acrylic copolymer dispersions sold under the names Neocryl XK-90® (INCI name: acrylic/styrene copolymer), Neocryl A-1070® (INCI name: acrylic/styrene copolymer), Neocryl A-1090® (INCI name: acrylic/styrene copolymer), Neocryl BT-62® (INCI name: acrylic/styrene copolymer), Neocryl A-1079® (INCI name: acrylic/styrene copolymer) and Neocryl A-523® (INCI name: acrylic/styrene copolymer) by the company Avecia-Neoresins, Dow Latex 432® (INCI name: Styrene/Acrylates Copolymer) by the company Dow Chemical, Daitosol 5000 AD® (INCI name: acrylates copolymer) by the company Daito Kasey Kogyo; or the aqueous dispersions of polyurethane sold under the names Neorez R-981® (INCI name: polyester-polyurethane copolymer) and Neorez R-974® (INCI name: polyester-polyurethane copolymer) by the company Avecia-Neoresins, Avalure UR-405® (INCI name: polyurethane-2), Avalure UR-410® (INCI name: polyurethane-2), Avalure UR-425° (INCI name: polyurethane-2), Avalure UR-450® (INCI name: PPG-17/IPDI/DMPA copolymer), Sancure 875® (INCI name: polyester-polyurethane copolymer), Sancure 861® (INCI name: polyester-polyurethane copolymer), Sancure 878® (INCI name: polyester-polyurethane copolymer) and Sancure 2060° (INCI name: polyester-polyurethane copolymer) by the company Goodrich, Impranil 85® (INCI name: Water and Polyurethane/Polyester) by the company Bayer and Aquamere H-1511® (INCI name: PVP/polycarbamyl/polyglycol Ester) by the company Hydromer. Aqueous polymer dispersions resulting from the radical-mediated polymerization of one or more radical-mediated monomers within and/or partially at the surface of pre-existing particles of at least one polymer chosen from the group consisting of acrylic copolymers, polyurethanes, polyureas, polyesters, polyesteramides and/or alkyds may also be used. These polymers are generally referred to as hybrid polymers.

The latex is present in the inventive compositions in amounts generally ranging from about 10% to about 60%, in some embodiments from about 20% to about 50%, and in some other embodiments from about 25% to about 40%, by weight, based on the total weight of the cosmetic composition.

### Filler

According to the present invention, compositions comprising a filler are provided. The fillers useful in the present invention may modify the texture of the composition. Representative examples of suitable fillers include polyamide powders, for instance nylon-12 and Nylon® (e.g., Orgasol from Elf Atochem), tetrafluoroethylene polymer powders, for instance Microslip 519 L (Micro Powders, Inc. (Tarrytown, N.Y.)) and Teflon®, silica, mica, kaolin, iron oxides, titanium dioxide, poly-alanine powders, polyethylene powders, starch, hollow polymer microspheres such as those of polyvinylidene chloride/acrylonitrile, for instance Expancel® (Nobel Industrie), acrylic powders such as Polytrap® (Dow Corning), polymethyl methacrylates particles and silicone resin microbeads (for example Tospearls® from Toshiba), magnesium hydrocarbonate, hydroxyapatite, hollow silica microspheres (Silica Beads® from Maprecos), and glass and ceramic microcapsules.

The filler is present in amounts generally ranging from about 10% to about 60%, in some embodiments from about 20% to about 50%, and in some embodiments from about 30% to about 45%, by weight, based on the total weight of the composition.

The ratio of latex to filler generally ranges from about 3.5:1 to about 1:1.5; in some embodiments from about 3:1 to about 1:1. In some other embodiments, the ratio of latex to filler is about 77:23 or about 55:45. In certain embodiments, the ratio of latex to filler is about 3:1, about 2.75:1, about 2.5:1, about 2.25:1, about 2:1, about 1.75:1, about 1.5:1, about 1.25:1, or about 1:1.25.

### Wax-Free

The cosmetic compositions of the present invention are free from any wax. For the purposes of the present invention, the term “wax” means a lipophilic fatty compound that is solid at room temperature about (25° C.) and atmospheric pressure (760 mmHg, i.e., 105 Pa), which undergoes a reversible solid/liquid change of state and which has a melting point of greater than 30° C. Waxes that are excluded from the present invention may be of animal origin, plant origin, mineral origin or synthetic origin. Examples of waxes of animal origin include beeswaxes, lanolin waxes and Chinese insect waxes. Examples of waxes of plant origin include rice waxes, carnauba wax, candelilla wax, ouricurry wax, cork fibre waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, waxes obtained by Fischer-Tropsch synthesis, waxy copolymers and their esters, and silicone and fluoro waxes. Waxes may also be hydrogenated oils of animal or plant origin.

### Optional Ingredients

### Thickener

The cosmetic compositions of the present invention may include a thickener. The thickeners include water-soluble thickeners and oil phase thickeners. Representative examples of suitable water-soluble thickeners include polyvinylpyrrolidone (PVP), polyvinyl alcohol, crosslinked acrylates (e.g. Carbopol 982), hydrophobically-modified acrylates (e.g. Carbopol 1382); polyacrylamides such as, for example, the crosslinked copolymers sold under the names Sepigel 305 (CTFA name: polyacrylamide/C13-C14 isoparaffin/Laureth 7) or Simulgel 600 (CTFA name: acrylamide/sodium acryloyldimethyltaurate copolymer/isohexadecane/polysorbate 80) by SEPPIC; 2-acrylamido-2-methylpropanesulphonic acid polymers and copolymers, that are optionally crosslinked and/or neutralized; cellulose derivatives such as hydroxyethylcellulose, sodium carboxymethylcellulose, hydroxypropyl methylcellulose, hydroxypropyl cellulose, ethyl cellulose and hydroxymethyl cellulose; polysaccharides and gums, e.g., natural gums such as xanthan gum (e.g., Rhodicare XC from Rhodia Inc. (Cranbury, N.J.), sclerotium, carrageenan and pectin; polysaccharide resins such as starch and its derivatives, hyaluronic acid and its salts, clays, and, in particular, montmorillonites, hectorites, bentonites, and laponites, crosslinked polyacrylic acids, such as the “Carbopol” products from the company Goodrich, the polyglyceryl (meth)acrylates polymers sold under the names “Hispagel” or “Lubragel” by the companies Hispano Quimica or Guardian, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare SC95” by the company Allied Colloid, and associative polymers and, in particular associative polyurethanes.

Representative examples of suitable oil phase thickeners include modified clays including hectorites modified with an ammonium chloride of a C₁₀ to C₂₂ fatty acid, such as hectorite modified with distearyldimethylammonium chloride, also known as quaternium-18 bentonite, such as the products sold or made under the names Bentone 34 by the company Rheox, Claytone XL, Claytone 34 and Claytone 40 sold or made by the company Southern Clay, the modified clays known under the name quaternium-18 benzalkonium bentonites and sold or made under the names Claytone HT, Claytone GR and Claytone PS by the company Southern Clay, the clays modified with stearyldimethylbenzoylammonium chloride, known as stearalkonium bentonites, such as the products sold or made under the names Claytone APA and Claytone AF by the company Southern Clay, and Baragel 24 sold or made by the company Rheox. Other mineral gelling agents include silica, such as fumed silica. The fumed silica may have a particle size ranging from about 5 nm to 200 nm.

In an embodiment of the present invention the thickener is xanthan gum and or PVP. A thickener, if present, is in amounts generally ranging from about 0.1% to about 3%, and in some embodiments, about 0.2% to about 2%, and in other embodiments, about 0.25%, about 0.50%, about 0.75%, or about 2%, by weight, based on the total weight of the composition.

### Solvent

The cosmetic compositions of the present invention may contain a solvent in addition to the water of the latex. The solvent may be aqueous or non-aqueous in nature, polar or non-polar, or volatile or non-volatile.

The solvent may or may not include additional water. Cosmetic compositions that are washable contain water. On the other hand, water may or may not be present in waterproof cosmetic compositions. Generally, water content of washable cosmetic compositions ranges from about 20 to about 80%, by weight, and in some embodiments from about 30 to about 60%, by weight, based on the total weight of the composition. In contrast, water content of waterproof cosmetic compositions generally ranges from 0 or greater than 0 to about 60%, by weight, and in some embodiments from 0 or greater than 0 to about 35%, by weight, based on the total weight of the composition. One or more water-miscible solvents (miscibility in water of greater than 50% by weight at about 25° C.) may also be present. Examples include lower monoalcohols containing from 1 to 5 carbon atoms such as ethanol and isopropanol, glycols containing from 2 to 8 carbon atoms, such as propylene glycol, ethylene glycol, butylene glycol or dipropylene glycol and pentylene glycol, C₃-C₄ ketones and C₂-C₄ aldehydes.

Representative examples of suitable volatile solvents include non-polar volatile hydrocarbon-based oils (which as used herein, refers to oil containing only hydrogen and carbon atoms), silicone oils (optionally comprising alkyl or alkoxy groups that are pendant or at the end of a silicone chain), and fluoro oils. Representative examples of suitable hydrocarbon-based oils include isoparaffins, i.e., branched alkanes containing 8-16 carbon atoms, such as isododecane (also known as 2,2,4,4,6-pentamethylheptane), and petroleum distillates, ethylhexyl palmitate, commercially available from Croda Chemicals under the tradename Crodamol OP, and dicaprylyl ether. Representative examples of suitable silicone oils may include linear or cyclic silicones containing from 2 to 7 silicon atoms, and which optionally contain alkyl or alkoxy groups containing from 1 to 10 carbon atoms. Examples include octamethylcyclotetrasiloxane, decamethylcyclopentasiloxane, hexadecamethylcyclohexasiloxane, heptamethylhexyltrisiloxane and heptamethyloctyltrisiloxane.

Representative examples of suitable polar volatile solvents include C₂ to C₅ alcohols, such as ethanol, ethyl 3-ethoxypropionate and isohexyl neopentanoate.

A volatile solvent, if present, is in amounts generally ranging from about 0.1% to about 80%, and in some embodiments, from about 5% to about 70%, and in other embodiments, from about 10% to about 60%, based on the total weight of the composition.

Exemplary non-volatile solvents include cosmetically or dermatologically acceptable and, in general, physiologically acceptable oils, such as carbon-based, hydrocarbon-based, fluoro and/or silicone oils, of mineral, animal, plant or synthetic origin, alone or as a mixture. Representative examples of suitable non-volatile solvents or oils include synthetic esters and ethers, especially of fatty acids, for instance the oils of formulae R¹COOR² and R¹OR² in which R¹ and R² represent, independently of each other, a branched or unbranched hydrocarbon-based chain containing from 3 to 30 carbon atoms, for instance purcellin oil, isononyl isononanoate, isopropyl myristate, ethylhexyl palmitate (commercially available from Croda Chemicals as Crodamol OP), 2-octyldodecyl stearate, 2-octyldodecyl erucate, isostearyl isostearate or isononyl isononanoate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate (Prisorine 3631); linear or branched hydrocarbons of mineral or synthetic origin, such as volatile or non-volatile liquid paraffins, and derivatives thereof. Representative examples of silicone oils are methicone phenyl trimethicone, commercially available from Dow-Corning as DC 556, and caprylyl methicone, commercially available from Dow-Corning as FZ-3196.

In some embodiments, the non-volatile solvent is a polyalphaolefin, which includes ethylene derivatives oligomerized into even-numbered carbon polyalphaolefins e.g., C₆-C₁₄ olefins such as polydecene and polymers of C₆, C₈, C₁₂ and C14 olefins. The polyolefins may have a molecular weight (MW) generally ranging from about 280 to about 11,500, and a viscosity (CPs at about 20° C.) generally ranging from about 7 to about 32,500. They may also be hydrogenated. In some embodiments, the non-volatile solvent includes PureSyn™ 2 (MW about 283), 4 (MW about 432), 6 (MW about 570), 8 (MW about 611), 150 (MW about 3980) and 300 (MW about 4870) (INCI name: hydrogenated polydecene). The viscosity of these polymers is about 8, about 33, about 64, about 103, about 4179 and about 8400, respectively.) PureSyn™ 100 (MW about 2939, viscosity about 3900, INCI name: hydrogenated C₆₋₁₄ olefin polymers) and PureSyn™ 1000 (MW about 11,500, viscosity about 32,400, INCI name: polydecene) may also be useful. The PureSyn™ products are commercially available from Exxon Chemicals.

The non-volatile solvent, if present, is in amounts generally ranging from about 0.1% to about 70%, and in some embodiments, about 0.5% to about 40%, and in other embodiments, 1% to about 25%, based on the total weight of the composition.

### Emulsifier

The compositions of the invention may also contain an emulsifier. Suitable emulsifiers include nonionic emulsifiers, cationic emulsifiers, amphoteric/zwitterionic emulsifiers, and anionic emulsifiers.

Representative examples of suitable nonionic emulsifiers useful in the compositions of the invention include fatty acids (e.g., C₁₂-C₁₆ fatty acids such as stearic acid), fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic emulsifiers, C₁₆-C₃₀ fatty acids neutralized by amines, ammonia or the alkali metal salts thereof.

Representative examples of suitable cationic emulsifiers include quaternary amines, amine oxides and amines, e.g., alkyl amines, alkyl imidazolines, ethoxylated amines, quaternary compounds, and quaternized esters. Cationic emulsifiers may also provide a conditioning effect.

Representative examples of suitable amphoteric emulsifiers include lauryl betaine, lauroamphoglycinate, lauroamphopropylsulfonate, lauroamphopropionate, lauroampho-carboxyglycinate, lauryl sultane, myristamidopropyl betaine, myristyl betaine, myristoamphoglycinate, myristyl propionate, stearoamphoglycinate, stearoamphopropionate, stearoamphopropylsulfonate, stearyl betaine, cocamidoethyl betaine, cocamidopropyl betaine, cocamidopropyl hydroxysultane, cocamidopropyl dimethyl amine propionate, cocoamphoglycinate, cocoamphocarboxypropionate, cocoamphocarboxyglycinate, coco-betaine, cocoamphopropionate, and cocoamphopropylsulfonate.

Representative examples of suitable nonionic emulsifiers include, fatty alcohols, such as cetyl alcohol, fatty acid esters, and alkoxylated, particularly ethoxylated, fatty acid esters of polyhydric alcohols such as glycerols and sorbitol, for example, polyoxyethylene monolaurate, polyoxyethylene monooleate, polyoxyethylene monostearate, sorbitan monolaurate, sorbitan trioleate, glyceryl stearate, steareth-12, ceteth-10, and PEG-100 stearate, generally with a degree of ethoxylation of from about 20 to about 85; mono- and di-alkanolamides, such as the N-acyl derivatives of mono- and di-ethanol amines, and polyethoxylated monoalkanolamides; amine oxides, such as cocoamidopropyl dimethylamine oxides, coco bis-2-hydroxyethyl amine oxides and lauryl dimmethylamine oxide; ethoxylated alkanolamides; ethoxylated oils and fats such as ethoxylated lanolins; ethoxylated alkylphenols, such as Nonoxynol, and polaxamers.

An emulsifier, if present, is in amounts generally ranging from about 1 to about 30% by weight, and in some other embodiments from about 3% to about 15% by weight, based on the total weight of the composition.

### Colorant

The inventive compositions may also contain a colorant, e.g., pigment. Pigments may be chosen from the organic and/or mineral pigments known in the art. These pigments may be in the form of pigmentary powder or paste. They may be coated or uncoated. To the extent that a colorant has filler-like properties, or is itself also a filler, it may be included in the amount of filler present in the compositions of the present invention.

The pigments may be chosen, for example, from natural (mineral) pigments, organic pigments, and pigments with special effects such as nacres or glitter flakes.

Natural pigments may be chosen from, for example, iron oxides, mica (e.g., mica sericite), chromium oxides, manganese violet, ultramarine blue, titanium dioxide, chromium hydrate and ferric blue.

The organic pigment may be chosen from nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

White or colored organic pigments may be chosen from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

Examples that may also be mentioned include pigmentary pastes of organic pigments, such as the product sold by the company Hoechst under the names: Jaune Cosmenyl IOG: Pigment Yellow 3 (CI 11710); Jaune Cosmenyl G: Pigment Yellow 1 (CI 11680); Orange Cosmenyl GR: Pigment Orange 43 (CI 71105); Rouge Cosmenyl R: Pigment Red 4 (CI 12085); Carmine Cosmenyl FB: Pigment Red 5 (CI 12490); Violet Cosmenyl RL: Pigment Violet 23 (CI 51319); Bleu Cosmenyl A2R: Pigment Blue 15.1 (CI 74160); Vert Cosmenyl GG: Pigment Green 7 (CI 74260); Noir Cosmenyl R: Pigment Black 7 (CI 77266).

The pigments may also be in the form of composite pigments as described in EP 1 184 426. These composite pigments may be compounds of particles comprising a mineral core, at least one binder for ensuring the binding of the organic pigments to the core, and at least one organic pigment at least partially covering the core.

The organic pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Among the dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 0 (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform colored appearance (characterized by a certain shade, a certain vivacity and a certain lightness) that changes as a function of the conditions of observation (light, temperature, observation angles, etc.). They thus contrast with white or colored pigments that afford a standard uniform opaque, semi-transparent or transparent shade.

Several types of pigment with special effects exist: those with a low refractive index, such as fluorescent, photochromic or thermochromic pigments, and those with a high refractive index, such as nacres or glitter flakes.

Examples of pigments with special effects that may be mentioned include nacreous pigments such as white nacreous pigments such as mica coated with titanium or with bismuth oxychloride, colored nacreous pigments such as titanium mica with iron oxides, titanium mica with ferric blue or with chromium oxide, titanium mica with an organic pigment of the abovementioned type, and also nacreous pigments based on bismuth oxychloride. Nacreous pigments that may be mentioned include the Cellini nacres sold by Engelhard (mica-TiO₂-lake), Prestige sold by Eckart (mica-TiO₂), Prestige Bronze sold by Eckart (mica-Fe₂O₃), and Colorona sold by Merck (mica-TiO₂—Fe₂O₃).

In addition to nacres on a mica support, multilayer pigments based on synthetic substrates such as alumina, silica, sodium calcium borosilicate or calcium aluminum borosilicate, and aluminum, may be envisaged.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength ranging from 400 nm to 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

The variety of pigments that may be used makes it possible to obtain a wide range of colors, and also particular optical effects such as metallic effects or interference effects.

A colorant, such as a pigment, if present, is in amounts generally ranging from about 0.01% to about 50%, based on the total weight of the composition, and in some embodiments, about 0.5% to about 20%, and in other embodiments, 1% to about 10%, based on the total weight of the composition.

### Additional Polymers

The cosmetic compositions may contain additional polymers, e.g., film forming polymers that are compatible with the other ingredients and form a film after application. Suitable polymers include polyvinylpyrrolidones (PVP) and vinyl copolymers, e.g., vinyl pyrrolidone (VP)/hexadecane copolymer, PVP/hexadecene copolymer and VP/eicosene copolymer (e.g., Ganex V220, which is a trade name of ISP Inc. of Wayne, N.J.), trimethylsiloxysilicate and acrylates copolymer. An additional polymer, if present, is in amounts generally ranging from about 0.1% to about 20% by weight.

### Chelating Agent

The cosmetic compositions of the present invention may include a chelating agent. Representative examples of suitable chelating agents include EDTA and salts thereof, particularly sodium and potassium salts. A chelating agent, if present, is in amounts generally ranging from about 0.01% to about 5% by weight of the composition.

### Adhesive

The cosmetic compositions of the present invention may include an adhesive. A representative example of a suitable adhesive is polycyclopenadiene, such as Koboguard® 5400 AD, available from Kobo Products, Inc. (South Plainfield, N.J.). An adhesive, if present, is in amounts generally ranging from about 0.01% to about 15% by weight of the composition.

### Dispersion Enhancing Agent

The compositions of the present invention may also contain dispersion enhancing agents. Representative examples of suitable dispersion enhancing agents include lecithin and polysaccharide resins, such as KM 13, available from KAMA International Corporation (Duluth, Ga.). Dispersion enhancing agents are especially preferred in pigmented products. A dispersion enhancing, if present, is in amounts generally ranging from about 0.1% to about 5% by weight of the composition.

### Preservative

The cosmetic compositions of the present invention may also contain preservatives. Representative examples of suitable preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol. Mixtures of preservatives are commercially available, e.g., the mixture of methylparaben, ethylparaben, propylparaben and butylparaben sold under the name Nipastat by Nipa, the mixture of phenoxyethanol, methylparaben, ethylparaben, propylparaben and butylparaben, also sold by Nipa under the name Phenonip, and the mixture of phenoxyethanol, methylparaben, isopropylparaben, isobutylparaben and butylparaben, sold by ISP under the tradename Liquapar Optima. The preservative may further include a co-preservative such as caprylyl glycol. A preservative, if present, is in amounts generally ranging from about 0.01% to about 15% by weight of the composition.

The compositions of the present invention may further contain at least one further (e.g., cosmetically or dermatologically acceptable) ingredient, including additives and adjuvants, including, for example, waxes, moisturizers, colorants, polymers (other than the polymer of the latex), gelling activators, humectants, anti-foam agents (e.g., simethicone, which is a fluid composition containing polydimethylsiloxane and silica), sunscreen agents (e.g., inorganic sunscreen agent, such as titanium dioxide and zinc oxide and organic sunscreen agents, such as octocrylene, ethylhexyl methoxycinnamate, and avobenzone), fibers, antioxidants (e.g., BHT, tocopherol), essential oils, fragrances, and cosmetically active agents and dermatological active agents such as, for example, anti-inflammatory agents, vitamins, and trace elements. These ingredients may be soluble or dispersible in whatever phase or phases is/are present in the cosmetic composition (i.e., aqueous and/or fatty phase).

The cosmetic compositions of the present invention may be made by mixing the latex and the filler. Additional ingredients may be added before, during, or after the mixing of the latex and the filler. In an embodiment, the entire process for making the cosmetic composition is carried out without the introduction of additional heat. In one embodiment, the entire process for making the cosmetic composition is carried out at a temperature of about 50° C. or less. In a further embodiment, the entire process for making the cosmetic composition is carried out at a temperature of about 30° C. or less. In another embodiment, the entire process for making the cosmetic composition is carried out at a temperature of about room temperature (i.e., about 20° C. to about 25° C.) or less.

The compositions for application to keratinous tissue may constitute in particular a cosmetic or dermatological composition for protecting, treating, or caring for keratinous tissue, such as hair, including eyelashes and eyebrows, skin, and lips. In certain embodiments, the cosmetic compositions of the present invention are in the form of mascara, a foundation, an eye shadow, an eyeliner, a blusher, a lipstick, a lipgloss, a care base or balm for the lips, or a concealer product. The cosmetic compositions of the present invention may be applied to keratinous tissue by hand. Alternatively, or in conjunction therewith, they may be applied via an applicator such as a sponge, cotton, brush, or a puff of a natural or synthetic material. In addition, the applicator may be attached to or contained within a container that serves as a reservoir for the cosmetic composition.

It has been surprisingly found that compositions according to an embodiment of the present invention have excellent long wear, non-flaking, volume, and tubing properties, without the need for to employ any wax. The omission of the wax allows for manufacture of the cosmetic compositions without the specialized equipment and steps required to melt and disperse wax in the other ingredients. Moreover, the compositions may be formed at or near room temperature and without the need for any additional heating. Accordingly, the compositions of the present invention may be produced more easily, quickly, and cheaply than conventional compositions containing wax.

The following examples are intended to further illustrate the present invention. They are not intended to limit the invention in any way. Unless otherwise indicated, all parts are by weight.

### EXAMPLES

### Example 1

Cosmetic compositions in the form of a mascara containing latex, namely Daitosol® 5000 AD, one of three different fillers (i.e., Silica N20, Nylon-12, or PTFE), and 0.5% xanthan gum, were made by first mixing the latex and filler in a homogenizer and then adding the xanthan gum using a crusher blade. The making of the mascaras was carried out at about room temperature without any added heat. Four different ratios of latex to filler were prepared—100:0 (no filler), 85:15, 70:30, and 55:45.

Each mascara was tested for flaking, tubing, and volume and rated on a scale of 1 (very bad) to 4 (very good) for flaking, tubing, and volume on false eyelashes. The procedures and results are presented below.

### Flaking

Ten (10) strokes of the mascara to the false eyelashes. After one hour, a curl brush was run over the false eyelashes 60 times.

Flaking is a measure were applied of the flexibility and long wear of the mascara composition. Large amounts of flaking indicate that the mascara is inflexible and/or does not adhere well to the lashes. Thus, a very good rating of 4 was given when little or no flaking occurred.

The results are tabulated below.

| Ratio of | Filler/Rating | | |
|---|---|---|---|
| Latex:Filler | Silica 20N | Nylon-12 | PTFE |
| 100:0 | 3 | 3 | 3 |
| 85:15 | 4 | 4 | 3 |
| 70:30 | 3 | 2 | 3 |
| 55:45 | 3 | — | 4 |

### Tubing

Ten (10) strokes of the mascara were applied to the false eyelashes. After one hour, the false eyelashes were run under 35° C. water for 30 seconds. The lashes were then laid down on paper and cardboard placed on top of the lashes. The lashes were then dragged out from below the cardboard and the degree of removal (i.e., tubing) was evaluated.

Tubing is a measure of the propensity of the mascara to roll up and off of the lash when being removed. A very good rating of 4 was given when the mascara quickly and easily rolled up and off of the lash when removal was desired.

The results are tabulated below.

| Ratio of | Filler/Rating | | |
|---|---|---|---|
| Latex:Filler | Silica 20N | Nylon-12 | PTFE |
| 100:0 | 1 | 1 | 1 |
| 85:15 | 1 | 1 | 2 |
| 70:30 | 2 | 4 | 3 |
| 55:45 | 3 | — | 4 |

### Volume

Either fifteen (15) or thirty (30) strokes of the mascara were applied to the false eyelashes. After one hour, the false eyelashes were compared to commercially available mascaras for volume.

Volume is a measure of the apparent build up of the mascara on the lashes. As volume increases each lash is defined and stands out to the eye, creating the desirable impression of longer and fuller lashes. Thus, a very good rating of 4 was given for a mascara that shows a large increase in volume.

| Ratio of | Filler Rating | | |
|---|---|---|---|
| Latex:Filler | Silica 20N | Nylon-12 | PTFE |
| 100:0 | 1 | 1 | 1 |
| 85:15 | 1 | 1 | 2 |
| 70:30 | 1 | 3 | 3 |
| 55:45 | 2 | — | 4 |

Volume after 15 Strokes

| Ratio of | Filler Rating | | |
|---|---|---|---|
| Latex:Filler | Silica 20N | Nylon-12 | PTFE |
| 100:0 | 1 | 1 | 1 |
| 85:15 | 1 | 2 | 3 |
| 70:30 | 2 | 4 | 4 |
| 55:45 | 3 | — | 4 |

Volume after 30 Strokes

Generally, these results indicate lower ratios of latex to filler (i.e., 70:30 and 55:45) produced mascaras with good to very good flaking, tubing, and volume ratings. Higher ratios of latex to filler tended to produce mascaras with less volume and poorer tubing characteristics.

In sum, the flaking properties were roughly equivalent across all ratios of latex to filler. In contrast, the volume and tubing properties were noticeably better for lower ratios of latex to filler (i.e., 70:30 and 55:45).

### Example 2

Cosmetic compositions in the form of a mascara containing latex, namely Daitosol 5000 AD, one of three different fillers (i.e., Silica N20, Nylon-12, or PTFE), and xanthan gum, were made by first mixing the latex and filler in a homogenizer and then adding the xanthan gum using a crusher blade. Three different ratios of latex to filler were prepared—85:15, 70:30, and 55:45 and different levels of xanthan were used.

Each mascara was evaluated for its aesthetic properties and those observations are presented below.

Ratio of Latex to Silica 20N 85:15; 0.25% wt Xanthan

The silica did not stay dispersed. The film formed was tacky and not much volume was produced. The film stayed on the skin, but came off easily with water.

Ratio of Latex to Silica 20N 85:15; 0.5% wt Xanthan

The film formed was as tacky as the previous mascara, and produced slightly more volume. The mascara was much more viscous than the previous mascara. The composition dried quickly and was very aerated.

Ratio of Latex to Silica 20N 85:15; 0.75% wt Xanthan

The film formed was tacky, but much less tacky than the two previous mascaras. The film had a very smooth feel and came off of the skin very easily.

Ratio of Latex to Silica 20N 70:30; 0.25% wt Xanthan

The silica stayed dispersed. The film formed was not tacky. The mascara produced slightly more volume than the previous three mascaras and was not as aerated.

Ratio of Latex to Silica 20N 70:30; 0.5% wt Xanthan

The film formed was very smooth and had moderate volume. The mascara went onto the skin very smoothly and stayed on the skin for a very long time without flaking.

Ratio of Latex to Silica 20N 70:30; 0.75% wt Xanthan

The film formed on film paper was very bumpy, but was much smoother when applied to the skin. The film formed stayed on the skin, but came off easily with water. The film produced a large volume.

Ratio of Latex to Silica 20N 55:45; 0.25% wt Xanthan

The silica stayed dispersed. The film had good volume, but was slightly bumpy. Some clumping of silica was observed, but otherwise the film was uniform.

Ratio of Latex to Silica 20N 55:45; 0.5% wt Xanthan

The film formed on the skin was smooth. The film stayed on the skin very well, but came off easily with water.

Ratio of Latex to Silica 20N 55:45; 0.75% wt Xanthan

The film formed was very smooth on the skin and produced a huge amount of volume.

Ratio of Latex to Nylon-12 85:15; 0.5% wt Xanthan

The film formed took slightly longer than the silica film did to dry. The film was very aerated and very thin. The film was non-homogenous.

Ratio of Latex to Nylon-12 85:15; 0.25% wt Xanthan

The composition was clay-like and was easily moldable in the hands. The composition was too solid-like to form a film.

Ratio of Latex to Nylon-12 77:23; 0.5% wt Xanthan

The film formed was very, very smooth and was not streaky. The film was very homogenous and not clumpy. The film produced good volume.

Ratio of Latex to Nylon-12 70:30; 0.5% wt Xanthan

The film formed was very streaky, but produced a large volume. The composition was thick and viscous and could be held upside down with any fluid flowing out of it.

Ratio of Latex to Nylon-12 70:30; 0.25% wt Xanthan

The film formed was slightly non-homogenous, not very smooth, and very aerated. The film came off too easily with water (i.e., when held under running water the film fell off.) The film produced a large volume. The composition was mousse-like.

Ratio of Latex to Nylon-12 55:45; 0.25% wt Xanthan

The film formed was very homogenous. The film was very smooth, but very aerated. The film came off easily with water. The composition was mousse-like.

Ratio of Latex to PTFE 85:15; 0.5% wt Xanthan

The film formed was very tacky and had a few clumps. The film produced little volume. The composition was very aerated and very fluid-like.

Ratio of Latex to PTFE 70:30; 0.5% wt Xanthan

The film looked very homogenous, but felt slightly rough. The film produced good volume. The film felt smooth on the skin and came off easily with water.

Ratio of Latex to PTFE 55:45; 0.5% wt Xanthan

The film formed was very opaque. When applied to the skin, the film was very smooth and voluminous and stayed on the skin well. The composition was very viscous.

To summarize, the compositions of the present invention may employ a wide range of ratios of latex to filler and differing amounts of thickener. The choice of ratio and thickener amount directly affects the properties of the film formed in terms of tackiness, smoothness, wear, etc. Generally, a ratio of latex to filler of 85:15 resulted in a film that was tacky and/or a composition that had undesirable physical properties. Lower ratios generally produced better volume and aesthetic properties.

### Example 3

A cosmetic composition of the present invention in the form of a mascara is described below.

| PHASE | INGREDIENTS | AMOUNT (Wt %) |
|---|---|---|
| A | Acrylates Copolymer | 28.08 |
| | (Latex) | |
| | Water | 12.17 |
| | Lecithin | 1.00 |
| | Polycyclopentadiene | 3.00 |
| | Phenoxyethanol | 1.00 |
| | Butylene Glycol | 5.00 |
| | Disodium EDTA | 0.20 |
| | PVP | 1.50 |
| | Capryl Glycol | 0.20 |
| | Black Iron Oxide | 7.00 |
| B | Polytetrafluoroethylene | 30.35 |
| | Nylon-12 | 5.00 |
| C | Xanthan Gum | 0.50 |
| D | Denatured Alcohol | 5.00 |
| | | 100.00 |

The mascara composition described above was prepared as follows. The ingredients of Phase A were homogenized at 200 rpm for 60 minutes in a Rayneri Turbotest® homogenizer. The ingredients of Phase B were added under sweeps. The xanathan gum (Phase C) was added under a crusher blade and mixed for 15 minutes. The denatured alcohol (Phase D) was added under sweeps. All steps were carried out at room temperature.

The mascara was then applied to the eyelashes of a panel of 12 subjects. The mascara was tested for wear, smudging, and flaking 8 hours after application. All 12 panelists still had visible mascara on the lashes at 8 hours, indicating long wear. None of the 12 panelists observed smudging and only 5 panelists observed flaking at 8 hours. Accordingly, the mascara exhibited long wear with no smudging and little flaking.

### Example 4

The mascara of Example 3 was tested to compare the volume (i.e., thickness) of lashes before and after application of the mascara. The lashes tested were made from medium brown, European, “virgin” hair. Six lashes were used for the testing and each lash was composed of five hair fibers. Thus, a total of 30 fibers were tested for volume change.

First, the thickness of each fiber before application of the mascara was measured. Then fifteen (15) strokes of the mascara were applied to the eyelashes. The thickness of each fiber was again measured.

The average thickness was determined for the untreated and treated fibers. The application of the inventive mascara produced an average increase in volume of 381%. The increase in volume was determined to be statistically significant. Thus, it has been shown that a composition of the present invention provided increased volume that was as good as that of previously known mascaras.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising:
a) about 10% to about 60% of a latex, and
b) about 10% to about 60% of a filler;
wherein:
i) the ratio of latex to filler is from about 3.5:1 about 1:1.5, and
ii) the composition is free of wax;
wherein the percentages are by weight based on the total weight of the cosmetic composition.

2. The cosmetic composition of claim 1, wherein the latex is an acrylic dispersion.

3. The cosmetic composition of claim 1, wherein the latex is an acrylates copolymer dispersion.

4. The cosmetic composition of claim 1, wherein the filler is selected from polyamide powders, tetrafluoroethylene polymer powders, and silicas.

5. The cosmetic composition of claim 1, wherein the filler is selected from nylon-12, and polytetrafluoroethylene, and silica.

6. The cosmetic composition of claim 1, wherein the ratio of latex to filler is about 55:45.

7. The cosmetic composition of claim 1, further comprising a solvent, a thickener, a colorant, a chelating agent, an adhesive, a dispersion enhancing agent, and/or a preservative.

8. The cosmetic composition of claim 7, wherein the solvent is selected from water, butylene glycol, and ethanol.

9. The cosmetic composition of claim 7, comprising two thickeners which are xanathan gum and polyvinylpyrrolidone.

10. The cosmetic composition of claim 7, wherein the colorant is black iron oxide.

11. The cosmetic composition of claim 7, wherein the chelating agent is disodium EDTA.

12. The cosmetic composition of claim 7, wherein the adhesive polycyclopentadiene.

13. The cosmetic composition of claim 7, wherein the dispersion enhancing agent is lecithin.

14. The cosmetic composition of claim 7, wherein the preservative comprises phenoxyethanol and caprylyl glycol.

15. The cosmetic composition of claim 1, comprising about 28% of acrylates copolymer, about 35% of a combination of nylon-12 and polytetrafluoroethylene, a solvent, a thickener, a colorant, a chelating agent, an adhesive, a dispersion enhancing agent, and a preservative, wherein the percentages are by weight based on the total weight of the cosmetic composition.

16. A process for making a cosmetic composition comprising mixing together:
a) about 10% to about 60% of a latex, and
b) about 10% to about 60% of a filler;
wherein:
i) the ratio of latex to filler is from about 3.5:1 about 1:1.5, and
ii) the composition is free of wax;
wherein the percentages are by weight based on the total weight of the cosmetic composition.

17. The process of claim 16, wherein the entire process is carried out a temperature of about 30° C. or less.

18. The process of claim 17, further comprising mixing a thickener, a colorant, a solvent, a chelating agent, an adhesive, a dispersion enhancing agent, and a preservative with the latex and filler.

19. The process of claim 17, comprising mixing together, at a temperature of about 30° C. or less, about 28% of acrylates copolymer, about 35% of a combination of nylon-12 and polytetrafluoroethylene, a solvent, a thickener, a colorant, a chelating agent, an adhesive, a dispersion enhancing agent, and a preservative, wherein the percentages are by weight based on the total weight of the cosmetic composition.

20. A method of making up or enhancing the appearance of a keratinous substance comprising applying to the keratinous substance a wax-free cosmetic composition, comprising:
a) about 10% to about 60% of a latex, and
b) about 10% to about 60% of a filler;
wherein:
i) the ratio of latex to filler is from about 3.5:1 about 1:1.5, and
ii) the composition is free of wax;
wherein the percentages are by weight based on the total weight of the cosmetic composition.
